(19) United States
(12) Patent Application Publication
(10) Pub. No.: US 2023/0404037 A1
(43) Pub. Date: Dec. 21, 2023

(54) AUTOMATED HIGH-RESOLUTION PUPAE COUNTING DEVICE

(71) Applicants: CONSEJO SUPERIOR DE INVESTIGACIONES CIENTIFICAS, Madrid (ES); UNIVERSIDAD MIGUEL HERNANDEZ, Allcante (ES)

(72) Inventors: Roberto SANTORO, Alicante (ES); Maria DOMINGUEZ CASTELLANO, Alicante (ES); Victor Javier RODRIGUEZ MILAN, Alicante (ES)

(21) Appl. No.: 18/036,519

(22) PCT Filed: Oct. 30, 2020
(86) PCT No.: PCT/ES2020/070670

§ 371 Date: May 11, 2023

(51) Int. Cl. A01K 29/00; G06M 7/00; G06M 11/00; H04N 5/76; G06T 7/00; H04N 23/56

(52) U.S. Cl. CPC A01K 29/005; G06M 7/00; G06M 11/00; H04N 5/76; G06T 7/0002; H04N 23/56; A01K 67/0339

(57) ABSTRACT

Pupae counting device that allows the automatic quantification of the youth-adult transition in groups of flies, like Drosophila. It comprises a circular platform, one or more tube holders positioned on the platform, one or more sample tubes, inserted into each tube holder, and intended to contain the pupae, a camera for recording video and images, directed towards the platform, a central motor linked and rotating the platform in front of the camera, until a sample tube of interest is positioned in front of the camera, and an external motor positioned beside the platform, for rotating the sample tube of interest 360° over itself in front of the camera.

### CROSS-REFERENCE TO RELATED APPLICATIONS AND PRIORITY

This patent application claims priority PCT Application No. PCT/ES2020/070670 filed Oct. 30, 2020. This patent application is herein incorporated by reference in its entirety.

### OBJECT OF THE INVENTION

The object of the invention is a pupae counting device that allows the automatic quantification of the youth-adult transition in groups of insects, preferably Drosophila flies. It can quantify, in real time and in a precise and reliable manner, the time of development of larva-adult in studies that require monitoring of the real time of initiation of sexual maturity.

### BACKGROUND OF THE INVENTION

Sexual growth and maturation occur through a series of stages of development precisely timed, but also showing plasticity and adaptation in response to certain environmental conditions and endogenous factors, such as disease, poor nutrition, mutations or endocrine disruptors.

There is an enormous interest on clinical factors causing early initiation in the transition between the juvenile to adult stage. The delay or acceleration of this transition can have a negative and persistent impact affecting both the final size and fertility, and is a risk factor for obesity, and certain cancers.

The beginning of sexual maturation (called metamorphosis in insects, and its equivalent, puberty, in mammals) is the most critical transition in the life story of an organism, which maximizes its reproductive success and adaptation to the environment.

The key signal of the beginning of sexual maturation has not been identified in any animal species and as a consequence, the control of this process has been the subject of intense research for decades.

The onset of sexual maturation varies from individual to individual within a species. The age of onset of maturation or metamorphosis/puberty is influenced by environmental factors, such as changes in temperature, and/or length of day (seasons), nutrition, or genetic factors, endocrine disruption or disease.

The youthful organisms do not enter into sexual maturation until they are competent, and have acquired an adequate development and growth. Toxic substances in the environment such as plastic, pollution, infections, malnutrition, infections, and diseases such as tumours and unknown genetic factors can alter the age of puberty, and metamorphosis in insects.

The vinegar fly Drosophila melanogaster is one of the most used insects in research on growth, development, neurogenetics, and physiology of ‘puberty’. The rate of development is also an important parameter in studies of efficacy and/or toxicity of drugs and recently this fly has been used for high efficiency drug screening studies in pre-clinical models of neurodegenerative and cancer diseases.

Document WO2004/006854 describes a method for screening for the effect of a test on a population of biological specimens, preferably insects, comprising the steps of providing a population of specimens, administering at least one test agent to the population, creating a digitized movie showing the movements of members of the population and measuring at least one trait of members of the population with the effect of the test agent.

Document CN206033758U describes a rotation platform based automated-high resolution tracking device for monitoring the presence or absence of bacterial growth.

### DESCRIPTION OF THE INVENTION

The pupae counting device of the present invention allows the automatic quantification of the youth-adult transition in groups of insects, preferably Drosophila flies. It can quantify, in real time and in a precise and reliable manner, the time of development of larva-adult in studies that require monitoring of the real time of initiation of sexual maturity.

To this end, the device comprises a camera that records video and image, directed towards a platform. On the platform, one or more tube holders, preferably arranged in a circular pattern, hold one or more sample tubes. A central motor is linked to the platform, rotating it in front of the camera, until a sample tube of interest is positioned in front of the camera. An external motor is positioned next to the platform, rotating a sample tube of interest, which is the one closest to the camera. In this way, the sample tube of interest can rotate 360° over itself, allowing the camera to record its entire surface.

The device can also comprise bearings, positioned between the tube holders and the platform, which is preferably circular, allowing their smooth rotation.

In an aspect of the invention, the camera is connected to an external device that runs a software which, using the video and images recorded by the camera, is capable of automatically quantifying the time and number of animals contained in the sample tubes, in different stages of development of the pupae in the youth-adult transition.

The external device can also monitor parameters that can affect experimental results such as food quality, bacterial contamination, etc.

In another aspect of the invention, the device comprises a lighting module, with an infrared and/or white light, that illuminates the sample tubes, in order to recreate day and night.

Preferably, both the motors (central motor and external motor) and the lighting module comprise a housing that covers them with an internal insulator, in order to protect them from humidity and allowing a longer life span of the device.

If the experiment conditions need to be fully controlled, the device can be positioned inside of an incubator, with humidity and temperature sensor and controllers, or any other variable sensor and actuator that could be needed. In this way, all variables of interest for the pupae development can be monitored and controlled when needed.

In this sense, in studies that require monitoring the time of initiation of sexual maturity, the automated high-resolution pupae counting device, object of the invention, saves time and improves accuracy and reproducibility in comparison with manual and visual counting.

Moreover, the device has the following advantages:
it is robust and durable and can be used in an incubator,
it is protected against moisture,
its movements are smooth and its rotations do not interfere with trials or tests of insect behavior,
it can be adapted to other insects besides Drosophila melanogaster,
the lighting module allows for circadian cycle experiments by registering and counting flies for twenty-four hours (light and dark) or longer periods (days, weeks), and
it allows to carry out development time experiments in highly controlled environmental conditions (e.g., humidity level, light, temperature, etc.).

The device can be adapted for high-efficiency experiments, such as the detection of genes, inhibitors, drugs or medicines, which need to count a lot of animals, accurately measure the time of death of the animal, or time its development from the juvenile to the adult stage with great precision.

### DESCRIPTION OF THE DRAWINGS

To complement the description being made and in order to aid towards a better understanding of the characteristics of the invention, in accordance with a preferred example of practical embodiment thereof, a set of drawings is attached as an integral part of said description wherein, with illustrative and non-limiting character, the following has been represented:

FIG. 1.—Shows a general view of the automated high-resolution pupae counting device.

FIG. 2.—Shows an upper view of the device.

### PREFERRED EMBODIMENT OF THE INVENTION

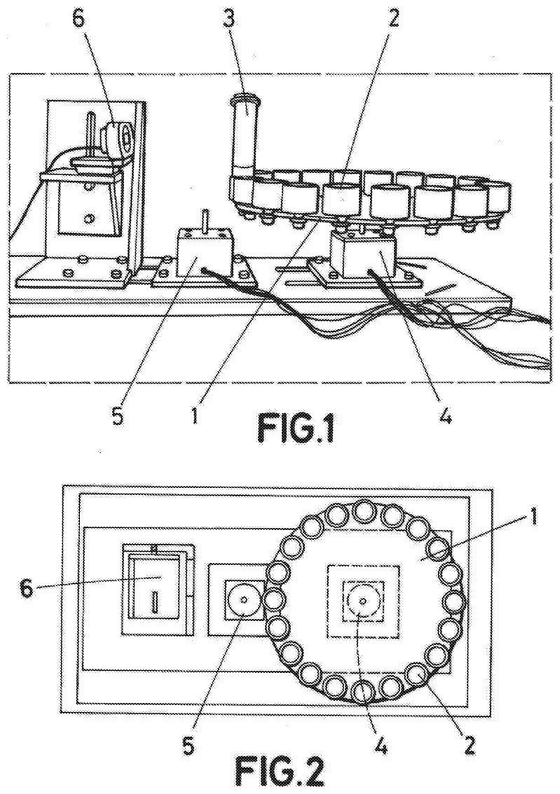

With the help of FIGS. 1 and 2, a preferred embodiment of the automated high-resolution pupae counting device, object of the present invention, is described below.

The device, shown in a general view in FIG. 1, is intended for automated monitoring and counting of Drosophila development. It allows an automated high-resolution monitoring and counting of larvae and pupae over days to weeks. The device can automatically count hundreds of flies during weeks in a single experiment.

The device comprises a camera (6) for recording video and images, directed towards a circular platform (1), as shown in FIG. 1. On the platform (1), one or more tube holders (2) are arranged in a circular pattern, as shown in FIG. 2. A sample tube (3), where the pupas are placed, is inserted into each tube holder (2).

A central motor (4) is linked to the platform (1), rotating it in front of the camera (6), until a sample tube (3) of interest is positioned in front of the camera (6). An external motor (5) is positioned besides the platform (1), rotating the sample tube (3) of interest, which is the one closest to the camera (6). In this way, the sample tube (3) of interest can rotate 360° over itself, allowing the camera (6) to record its entire surface, and therefore all the pupae contained inside.

The central motor (4) and the external motor (5) are stepper motors, facilitating the angular rotation control of both the platform (1) and the sample tubes (3).

The device also comprises bearings, not shown in FIG. 1, positioned between the sample tubes (3) and the platform (1), and that allow their smooth rotation. The slow movements and the low noise are ideal for the recording of multiple adult-fly genotype. The flies do not notice a thing.

The camera (6) is connected to an external device that, using the video and images recorded by the camera (6), runs a software capable of automatically quantifying the time and number of pupae in different stages of development in the youth-adult transition contained in each sample tube (3). The external device also monitors parameters that can affect experimental results such as food quality, bacterial contamination, etc. For example, food quality can be determined by its color.

The device additionally comprises a lighting module, with an infrared and a white light, that illuminates the sample tubes (3), in order to recreate day and night. The lighting module allows to perform circadian clock experiments.

Both the motors (4, 5) and the lighting module comprise a housing that covers them with an internal insulator, in order to protect them from humidity and allowing a longer life span of the device.

To allow a better adaptability to any kind of experiment, the sample tubes (3) can be any standard fly food vial/tube, not requiring special lab equipment.

The embodiment shown in FIG. 2 allows the recording of up to twenty sample tubes (3), with the possibility to increase this number for high-efficiency experiments.

1. An automated high-resolution pupae counting device that comprises:
a platform,
one or more tube holders arranged on the platform,
one or more sample tubes, inserted into each tube holder, and intended to contain pupae,
a camera for recording video and image, directed towards the platform (1), connected to an external device that quantifies the time and number of pupae contained in each sample tube,
a central motor linked to the platform,
being the device characterized in that the central motor rotates the platform until a sample tube of interest is positioned in front of the camera, and in that the device additionally comprises:
an external motor positioned beside the platform, that rotates the sample tube of interest 360° over itself in front of the camera.

2. The device according to claim 1, wherein the platform is circular and the tube holders are arranged in a circle.

3. The device according to claim 1, wherein the central motor and/or the external motor are stepper motors.

4. The device according to claim 1, wherein it additionally comprises bearings, positioned between the tube holders and the platform.

5. (canceled)

6. The device according to claim 1, wherein it additionally comprises a lighting module, that illuminates the sample tubes.

7. The device according to claim 6, wherein the lighting module comprises an infrared light and a white light.

8. The device according to claim 6, wherein the lightings module comprises a housing for humidity protection.

9. The device according to claim 1, wherein the external motor and the central motor comprise a housing for humidity protection.
